(19) United States
(12) Patent Application Publication
(10) Pub. No.: US 2025/0186403 A1
(43) Pub. Date: Jun. 12, 2025

(54) CALCIMYCIN AS ANTIFUNGALS

(71) Applicant: ARIZONA BOARD OF REGENTS ON BEHALF OF THE UNIVERSITY OF ARIZONA, Tucson, AZ (US)

(72) Inventors: Hongmin Li, Tucson, AZ (US); Zhong Li, Tucson, AZ (US); Qing-Yu Zhang, Tucson, AZ (US)

(21) Appl. No.: 18/844,645

(22) PCT Filed: Mar. 7, 2023
(86) PCT No.: PCT/US23/63850

§ 371 Date: Sep. 6, 2024

Related U.S. Application Data
(60) Provisional application No. 63/317,801, filed on Mar. 8, 2022.

(51) Int. Cl. A61K 31/423; A61P 31/10

(52) U.S. Cl. CPC A61K 31/423; A61P 31/10

(57) ABSTRACT

Pathogens developing resistance to available drugs are of main concern in various diseases, including cryptococcosis and tuberculosis. Hence there is a need for therapeutics with novel mechanisms of action. Described herein are compositions and methods for treating infections (e.g., fungal infections) caused by microbes (e.g., fungi) comprising inteins. An intein is a moving protein element inside a host protein. Splicing off intein from the host protein is required to maturate and activate the host protein. Calcimycin (CMN) is used as an intein splicing inhibitor to block intein splicing of the Prp8 protein of fungal pathogen C. neoformans and C. gattii the causative agents of cryptococcosis, to treat patients.

### CROSS-REFERENCES TO RELATED APPLICATIONS

This application claims benefit of U.S. Provisional Application No. 63/317,801 filed Mar. 8, 2022, the specification of which is incorporated herein in their entirety by reference.

### STATEMENT REGARDING FEDERALLY SPONSORED RESEARCH OR DEVELOPMENT

This invention was made with government support under Grant No. Al141178 and Al140726, awarded by National Institutes of Health. The government has certain rights in the invention.

### REFERENCE TO AN ELECTRONIC SEQUENCE LISTING

The contents of the electronic sequence listing (ARIZ_22_08_PCT_Sequence_Listing.xml; Size: 13,865 bytes; and Date of Creation: Feb. 28, 2023) is herein incorporated by reference in its entirety.

### FIELD OF THE INVENTION

The present invention features compositions and methods that treat diseases caused by pathogens, specifically pathogens comprising inteins.

### BACKGROUND OF THE INVENTION

Fungal infections affect more than 300 million people worldwide. Major fungal diseases include candidiasis, cryptococcosis, aspergillosis, and pneumocystosis. Other fungal diseases such as coccidioidomycosis (valley fever), histoplasmosis, and blastomycosis are also prevalent in certain parts of the world. Opportunistic fungal pathogens are one of the major causes of death of individuals with immunocompromised conditions caused by various reasons such as infections, transplantations, overuse of immunosuppressants, malnutrition, and radiotherapy. Each year approximately 223,100 human patients are affected by cryptococcosis meningitis globally, with 180,000 deaths. The cryptococcosis caused by Cne and Cga mainly affects the lung in the initial stage, leading to pneumonia-like conditions, followed by spreading to the brain, causing meningitis and mortality.

Four classes of antifungals are currently used as first-line drugs in the clinic. Polyenes, including Amphotericin B (AmB), affect fungal membranes via ergosterol binding. Azoles such as fluconazole (Flu), voriconazole, posaconazole, and the recently licensed isavuconazole inhibit ergosterol biosynthesis. Flucytosine, also known as 5-flucytosine (5-Fc), is a pyrimidine analog that can be converted to 5-fluorouracil in fungal cells, leading to the inhibition of fungal DNA and RNA synthesis. Echinocandins, including caspofungin, inhibit β-(1,3)-glucan synthase.

Frontline therapy in cryptococcal meningitis or severe pulmonary cryptococcosis uses AmB alone or combined with 5-Fc or Flu. However, increasing reports indicate that drug resistance and lesser drug permeability to the brain-blood barrier make the treatment less effective. Hence alternative targets and drugs are required to combat fungal diseases effectively.

### BRIEF SUMMARY OF THE INVENTION

It is an objective of the present invention to provide compositions and methods that allow for treating diseases caused by pathogens, specifically pathogens comprising inteins, as specified in the independent claims. Embodiments of the invention are given in the dependent claims. Embodiments of the present invention can be freely combined with each other if they are not mutually exclusive.

Alternative drug targets are necessary to overcome drug resistance before it attains a critical stage. Splicing of inteins from proprotein precursors is crucial for activities of essential proteins hosting intein elements in many organisms, including human pathogens such as Cne and Cga. Inteins are self-splicing protein elements found in the host precursor proteins. During the splicing process, inteins can ligate the host protein domains called exteins to form a mature active protein. Inteins are present in proteins of many organisms, including bacterial and fungal human pathogens. If the activity of a protein hosting inteins is crucial for the survival of an organism, inhibiting such a protein activity by blocking intein splicing will be detrimental to the pathogen. The pre-mRNA processing factor 8 (Prp8) proteins of Cne and Cga have an intein element. Prp8 is an integral part of the spliceosome complex involved in mRNA splicing. The size of the intein varies, from 171 amino acids (aa) in Cne to 819 aa in Aspergillus fumigatus (Afu), depending on the presence and absence of homing endonuclease. The presence or absence of inteins is species-specific. In the Cryptococcus genus, Cne and Cga have similar Prp8-inteins, whereas closely related C. amylolentus (Cam) does not have inteins.

The present invention features a high throughput screening (HTS) assay developed against wild-type Cne-H99 (Cne-WT) and in its inteinless mutant strain (Cne-Mut) using resazurin. Calcimycin (CMN) was identified as a potent compound, showing a minimum inhibitory concentration (MIC) of 1.5 μg/ml against Cne-WT, whereas that against Cne-Mut was 16-fold higher. CMN showed inhibitory activity on intracellular infection of Cne-WT in macrophages. The specificity of CMN towards the target was confirmed by a protein-based intein splicing inhibition assay employing split nanoluciferase-intein fusion protein with an IC₅₀ of 4.6 μg/ml. The binding of CMN to recombinant intein was demonstrated by thermal shift assay (TSA) and mesoscale thermophoresis (MST). CMN was found to inhibit intein splicing in vitro in split GFP-Prp8 intein assay and in vivo in Cne-WT. CMN was fungistatic and showed a synergistic effect with the known antifungal drug amphotericin B (AmB). In mice, the absorption and release of CMN were slow when given orally. The Tmax and t_{1/2} of CMN were 9.6 h and 7.2 h, respectively. Finally, CMN treatment at 20 mg/kg body weight (BW) led to a 60% reduction in lung-fungal load in a cryptococcal pulmonary infection mouse model. Overall, CMN represents a potent antifungal with a novel mechanism of action to treat Cne and Cga infection.

In some embodiments, the present invention features a method of treating a disease caused by microbes (e.g., pathogens) comprising inteins. The method comprises administering a therapeutically effective amount of calcimycin (CMN) to the subject. In other embodiments, the present invention may further feature a method of treating a fungal infection caused by a fungus in a subject in need thereof. The method comprises administering a therapeutically effective amount of calcimycin to the subject.

In some embodiments, the present invention features a method of inhibiting intein splicing in a microbe (e.g., a pathogen) comprising inteins, the method comprising introducing to said microbe a composition comprising calcimycin.

In some embodiments, the present invention features a composition comprising calcimycin for use in a method for the treatment of a disease caused by microbes (e.g., pathogens) comprising inteins. In other embodiments, the present invention features a composition comprising calcimycin for use in a method for the treatment of a fungal disease caused by a fungus comprising inteins.

One of the unique and inventive technical features of the present invention is the use of calcimycin to treat diseases caused by pathogens. Without wishing to limit the invention to any theory or mechanism, it is believed that the technical feature of the present invention advantageously provides for inhibition of intein splicing in an essential protein (e.g., Prp8), causing the organism not to survive. None of the presently known prior references or work has the unique, inventive technical feature of the present invention.

Any feature or combination of features described herein are included within the scope of the present invention provided that the features included in any such combination are not mutually inconsistent as will be apparent from the context, this specification, and the knowledge of one of ordinary skills in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show the effect of CMN on intracellular Cne. FIG. 1A shows the structure of CMN. FIGS. 1B, 1C, and 1D show phagocytosis assays. RAW 264.7 cells were seeded in 24 well plates and differentiated into macrophages using PMA. Media was replaced with fresh media containing opsonized Cne-WT and incubated at 37° C. for 8 h. Cells were washed 3 times with PBS to remove non-phagocytosed fungal cells. Media containing various concentrations of CMN were added and incubated for 24 h. Culture supernatants were diluted and plated onto SD agar plates for tittering the fungal load. The cells were washed thrice with PBS and homogenized with a small homogenizer, diluted, and plated on SD agar plates. Colonies were counted after 72 h. FIG. 1B shows fungal load in RAW 264.7 macrophage lysate. FIG. 1C shows fungal load in the culture supernatant. Treatment groups were compared with unpaired Student's t-test. p<0.05 is considered significant. FIG. 1D shows representative figures showing phagocytosed Cne inside the RAW 264.7 macrophages in different treatment groups. Insert: enlarged view of the phagocytosed Cne cells. The cells were permeabilized with methanol and stained with Giemsa stain. CFU: Counts per unit Cne: Cryptococcus neoformans.

FIGS. 2A, 2B, 2C, and 2D show CMN inhibits intein splicing. FIGS. 2A and 2B show a split-NanoLuc-Prp8 intein splicing assay: With a 1 nM final concentration of Split-NanoLuc-Prp8 intein protein, varying concentrations of the cisplatin (FIG. 2A) and CMN (FIG. 2B) was incubated for 30 min at RT in 96 well white opaque plates. TCEP is added to a final concentration of 100 UM and incubated for 24 h. The substrate Nano-Glo luciferase (Promega) was added, and the resulting luminescence was read. IC₅₀ was calculated using GraphPad prism. FIG. 2C shows a cf-split GFP-Prp8 intein splicing assay. The recombinant Cga split-cfGFP-Prp8 intein protein at a final concentration of 1.2 mg/ml was incubated with various concentrations of the CMN for 30 min at RT in a 1.5 ml centrifugation tube. TCEP was added to a concentration of 1 mM and incubated for 24 h. From this reaction mixture, 10 μl was mixed with an equal volume of 2×SDA-PAGE loading dye and loaded onto 12% SDS-PAGE gel without boiling the sample. The gel was scanned with UV using Chemidoc (Bio Rad), and band intensities were quantified using Image Lab software. FIG. 2D shows a western blot for in vivo intein splicing in Cne. Cne-WT (0.1 Abs at 600 nm) was incubated with various concentrations of CMN for 18 h. Cells were harvested and washed in PBS, lysed with bead lyser, and centrifuged at 10,000 rpm. An equal quantity of protein (50 μg) from each treatment was loaded onto 12% SDS-PAGE and blotted onto the NC membrane, blocked with 5% skimmed milk. The membrane was probed with anti-prp8 intein rabbit polyclonal sera, followed by goat anti-rabbit HRP antibody. Signals were developed using a chemiluminescent kit. The density of the spliced intein bands (˜20 kDa) were quantified using Image lab software. A band at ˜130 kDa is uncharacterized, as explained below. NC: nitrocellulose. Abs: absorbance

FIGS. 3A, 3B, and 3C show CMN specifically binds to Prp8 intein. FIG. 3A shows a ligand binding-TSA. The reaction mixture comprises 3 μM Cne prp8-intein protein, 1.27 UM CMN, Sypro orange fluorescent stain in 50 μl PBS pH 7.4. The Tₘ values were calculated by Thermal shift software. The graphs were drawn after normalizing relative fluorescence units using GraphPad Prism. FIG. 3B shows a ligand binding-MST. The recombinant protein Cga-Prp8 intein was labeled with His-Tag-labeling kit-Red-Tris-NTA (Monolith), incubated with various concentrations of the CMN (max 300 UM) and loaded to the MST instrument. The assay was repeated three times. Data were analyzed with MO. Affinity analysis software (nanoTEMPER Technologies). FIG. 3C shows the effect of CMN on Hh cholesterolysis: The recombinant Drosophila Hh protein having FRET component (C-Hh-Y) was used for the assay. The reaction volume was 50 μl, and the final components were assay buffer (50 mM bis tris, 5 mM EDTA, 100 mM NaCl, 0.4% Triton X-100), 30 UM C-Hh-Y, DMSO, or varying concentrations of CMN (6.25 to 200 UM). After incubating at RT for 30 min, cholesterolysis was induced by adding cholesterol (dissolved in ethanol) to a final concentration of 200 UM. After incubating at RT for 22 h, an aliquot was taken for reducing SDS-PAGE, followed by coomassie blue staining. The gel image was taken using ChemiDoc imaging system (Bio Rad) and band densities were analyzed using Image Lab software. Treatment groups were compared with Student's t-test. p<0.05 was considered as significant. Molecular weights C-Hh-Y: 80 kDa, Hh-Y: 52 kDa, C: 27 kDa, TSA: Thermal shift assay, Hh: Hedgehog, MST: Mesoscale thermophoresis.

FIGS. 4A, 4B, and 4C show in vitro and in vivo toxicity. FIGS. 4A and 4B show cell cytotoxicity of CMN. A549 and RAW 264.7 cells were grown in 96 well plates till 70% confluent. RAW 264.7 cells were differentiated into macrophages using PMA. Media was replaced by fresh media containing various concentrations of CMN or DMSO and incubated for 48 h in a CO₂ incubator. The cell viability was estimated by Cell Counting Kit-8 (GlpBio, CA, USA), and absorbance was read at 450 nm using microplate reader Synergy H1 (BioTek). CC₅₀ was calculated by GraphPad prism. FIG. 4A shows cell cytotoxicity in A549 cells. FIG. 4B shows cell cytotoxicity in RAW 264.7 macrophage cells. Abs: absorbance. FIG. 4C shows a toxicity study in mice. Adult BALB/c mice (n=2, male: female; 1:1) were dosed with CMN or vehicle control (VC) by oral gavage once daily for seven days. Bodyweight and other parameters were monitored daily (Table 3).

FIGS. 5A and 5B show the pharmacokinetic and efficacy of CMN in mouse models. FIG. 5A shows in vivo clearance of CMN. Adult C57BL/6 mice (n=5) were administered with CMN by oral gavage at 40 mg/kg. Blood samples were collected at 0, 0.5, 1, 2, 4, 8, 12, 16, 24, and 36 h after oral gavage. The plasma concentration of CMN and the internal standard were analyzed by LC-MS/MS. Other PK parameters are in Table 4. The values represent means±SD. FIG. 5B shows the efficacy of CMN. BALB/c mice were dosed with CMN 20 mg/kg orally daily once for 5 days. On day 0, mice were infected with H99 (10⁷ cells/mice) intranasally or mock-infected with PBS. Mice were euthanized on day 5, and lungs were dissected out, homogenized in RPMI-1640-MOPS media, and plated onto Sabouraud dextrose agar plates containing antibiotics. Plates were incubated at 30° C. for 72 h, and colonies were counted. Treatment groups were compared with unpaired Student's t-test. p<0.05 was considered as significant. VC: Vehicle control, (90% corn oil, 10% DMSO). Dpi: Days post-infection. CFU: Colony-forming units.

FIG. 6 shows the MFC of CMN in C. neoformans. The assay was performed by microdilution method as explained in materials and method. MIC was the minimum concentration at which there is no growth when observed visually after 48 h incubation. Cells in the wells were mixed by pipetting, and 30 μl of the culture taken from each well was plated onto SD agar plates. The plates were observed for colonies after 72 h. The MFC is the minimum drug concentration where there are no colonies. If the ratio of MFC/MIC≤4 is considered fungicidal.

FIG. 7 shows the combinational effect of CMN: A checkerboard assay was done by diluting the test compound and known drugs in RPMI-MOPS media. After adding Cne cells, the plates were incubated for 48 h. Well contents were mixed by pipette, and absorbance was read at 630 nm. MIC is defined as the minimum concentration of the drug to get 90% inhibition of absorbance. Based on the FIC, the combinatorial effects are decided as in Table 2.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. The term does not denote a particular age or sex. Thus, adult and newborn subjects, as well as fetuses, whether male or female, are intended to be included. In one embodiment, the subject is a mammal (e.g., a human) having a disease, disorder, or condition described herein. In another embodiment, the subject is a mammal (e.g., a human) at risk of developing a disease, disorder, or condition described herein. A “patient” is a subject afflicted with a disease or disorder. The term “patient” includes human and veterinary subjects. In certain instances, the term patient refers to a human.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The term “effective amount” as used herein refers to the amount of a therapy (e.g., a calcimycin) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., cryptococcosis or tuberculosis), disorder or condition, reduction or amelioration of the recurrence, development or onset of a given disease, disorder or condition, and/or to improve or enhance the prophylactic or therapeutic effect(s) of another therapy. In some embodiments, “effective amount,” as used herein also refers to the amount of therapy provided herein to achieve a specified result.

As used herein, and unless otherwise specified, the term “therapeutically effective amount” of calcimycin is an amount sufficient to provide a therapeutic benefit in the treatment or management of a disease caused by microbes (e.g., cryptococcosis or tuberculosis) or to delay or minimize one or more symptoms associated with a disease caused by microbes (e.g., cryptococcosis or tuberculosis). A therapeutically effective amount of calcimycin means an amount of therapeutic agent, alone or in combination with other therapies, which provides a therapeutic benefit in the treatment or management of a disease caused by microbes (e.g., cryptococcosis or tuberculosis). The term “therapeutically effective amount” can encompass an amount that improves overall therapy, reduces or avoids symptoms or causes, or enhances the therapeutic efficacy of another therapeutic agent.

The terms “administering” and “administration” refer to methods of providing a pharmaceutical preparation to a subject. Such methods are well known to those skilled in the art and include, but are not limited to, administering the compositions intranasally, parenterally (e.g., intravenously and subcutaneously), by intramuscular injection, by intraperitoneal injection, intrathecally, transdermally, extracorporeally, topically, orally or the like.

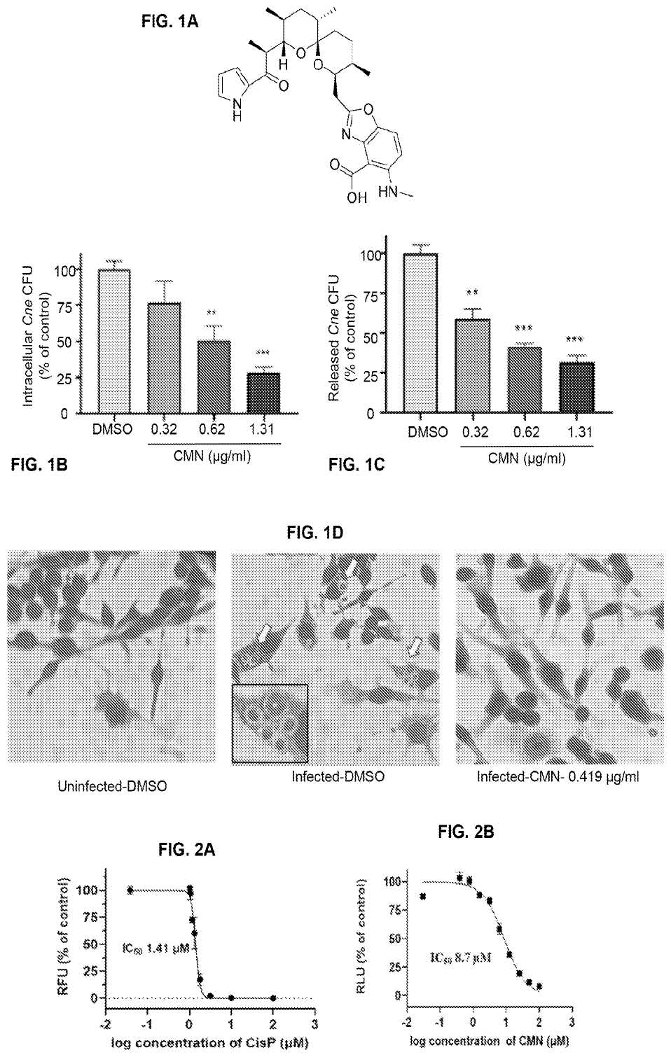

Referring now to FIGS. 1A-7, the present invention features composition and method for treating a disease and/or infection (e.g., fungal infections) caused by a microbe (e.g., fungus) comprising inteins.

The present invention features a method of treating a disease caused by a microbe (e.g., a pathogen) comprising inteins. The method comprises administering a therapeutically effective amount of calcimycin to the subject. In some embodiments, the present invention features a method of treating a disease caused by a fungus in a subject in need thereof. The method comprises administering a therapeutically effective amount of calcimycin to the subject.

In some embodiments, the microbe is a fungus. In other embodiments, the microbe is a bacterium. In some embodiments, the fungus comprises inteins. In some embodiments, the bacterium comprises inteins. In some embodiments, the fungus comprises proteins (e.g., Prp8) that comprise inteins (e.g., at least one intein). In some embodiments, the bacterium comprises proteins that comprise inteins (e.g., at least one intein).

In further embodiments, the microbe is a virus. In other embodiments, the microbe is an archaeon. In some embodiments, the virus comprises inteins. In some embodiments, the archaeon comprises inteins.

In some embodiments, the fungus is from the genus Cryptococcus. In some embodiments, the fungus is Cryptococcus neoformans (Cne). In some embodiments, the fungus is Cryptococcus gattii (Cga). In some embodiments, the disease is cryptococcosis. In other embodiments, the fungus is from the genus Candida. In some embodiments, the fungus is Candida fermentati, Candida parapsilosis, Candida krusei, or Candida albicans. In some embodiments, the disease is candidiasis.

In some embodiments, calcimycin (CMN) inhibits pre-mRNA processing factor 8 (Prp8) proteins. Without wishing to limit the present invention to any theory or mechanism, it is believed that CMN inhibits Prp8 splicing.

In some embodiments, the bacteria are from the genus Mycobacterium. In some embodiments, the bacteria is Mycobacterium tuberculosis (Mtb). In some embodiments, the disease is tuberculosis.

The present invention may further feature a method of treating a fungal infection caused by a fungus (e.g., a fungus comprising inteins) in a subject in need thereof. The method comprises administering a therapeutically effective amount of calcimycin to the subject. In some embodiments, the fungal infection is caused by Cryptococcus neoformans (Cne), Cryptococcus gattii (Cga), Candida fermentati, Candida parapsilosis, Candida krusei, or Candida albicans. In some embodiments, the fungal infection is cryptococcosis or candidasis.

In some embodiments, calcimycin is administered orally. In other embodiments, calcimycin is administered via an intravenous (IV) injection. In further embodiments, calcimycin is administered via an intramuscular (IM) injection.

The present invention may feature a composition comprising calcimycin for treating a disease caused by microbes (e.g., a pathogen) comprising inteins. In some embodiments, the present invention features a composition comprising calcimycin for use in a method for the treatment of a disease (e.g., the fungal infection) caused by a fungus comprising inteins. In some embodiments, the disease (e.g., the fungal infection) is caused by Cryptococcus neoformans (Cne), Cryptococcus gattii (Cga), Candida fermentati, Candida parapsilosis, Candida krusei, or Candida albicans. In some embodiments, the disease (e.g., the fungal infection) is cryptococcosis or candidasis.

In some embodiments, the composition comprising calcimycin is administered orally. In other embodiments, the composition comprising calcimycin is administered via an intravenous (IV) injection. In further embodiments, the composition comprising calcimycin is administered via an intramuscular (IM) injection.

The present invention may also feature a method of inhibiting intein splicing in a microbe (e.g., a pathogen) comprising inteins. The method comprises introducing to said microbe (e.g., said pathogen) a composition comprising calcimycin. In some embodiments, the present invention features a method of inhibiting intein splicing in a fungus comprising inteins. For example, calcimycin may inhibit the splicing of pre-mRNA processing factor 8 (Prp8) proteins in a microbe (e.g., a fungus).

The present invention may further feature the use of a composition comprising calcimycin for the manufacture of a medicament for the treatment of a disease (e.g., a fungal infection) caused by a microbe (e.g., a pathogen). In some embodiments, the present invention features the use of a composition comprising calcimycin for the manufacture of a medicament for the treatment of a disease (e.g., a fungal infection) caused by a fungus.

### Example

The following is a non-limiting example of the present invention. It is to be understood that said example is not intended to limit the present invention in any way. Equivalents or substitutes are within the scope of the present invention.

Chemicals and Reagents: Human Complement sera (S-1764, Sigma, USA), Anti-Glucuronoxylomannan clone 18B7 (MABF2069, EMD Millipore, USA), Phorbol 12-myristate 13-acetate (P8139, Sigma). Sabouraud Dextrose broth (BD, 238230), Nano-Glo-Luciferase (Promega N1110), Nano-Glo luciferase (Promega N110). Dulbecco's modified Eagle's media-high glucose DMEM (Sigma, D6429). Tris (2-carboxyethyl) phosphine hydrochloride TCEP (Sigma, C4706). Goat anti-rabbit IgG-HRP (Sigma, A-6154).

Cell culture: The human lung adenocarcinoma alveolar basal epithelial cells (A549) were purchased from ATCC, and grown in DMEM with 10% FBS in a CO₂ incubator at 37° C. To harvest the cells, 0.05% trypsin with 0.02% EDTA was used. The murine macrophage cells (RAW 264.7) were purchased from ATCC, and grown in DMEM with 10% FBS. The Cne-WT and inteinless mutant strains were grown at 30° C. either on the Sabouraud Dextrose (SD) agar plates or the SD broth in a shaker.

Mouse and study approval: BALB/c mice for toxicity and efficacy were purchased from Jackson lab. The protocols of efficacy study and toxicity study were approved by the Institutional Animal Care and Use Committee (IACUC), Wadsworth Center, Albany, NY, USA. C57BL/6 mice pharmacokinetic studies were obtained from breeding stocks maintained at the University of Arizona, Tucson, USA, and the protocol was approved by IACUC, University of Arizona.

Cloning, expression, and purification: All genes were codon-optimized, synthesized, and cloned into expression vectors by GeneUniversal.

For split nanoluciferase, the His and Myc-tagged CgaPrp8 intein sequence was inserted between nanoluciferase NanoBit residues 156 and 157 in the pET28 vector. The His- and Myc-tag (underlined) sequence GHHHHHHEQKLISEEDLG (SEQ ID NO: 1) was inserted between the prp8 intein residues 122 and 123 to facilitate purification. Similarly, for split GFP, the same His-Myc-tagged CgaPrp8 intein was placed between cysteine-free split GFP residues 128 and 129.

Unless otherwise specified, all proteins were expressed and purified according to the following general protocol. The plasmid was transformed into E. coli cell BL21 (DE3) competent cells. Colonies were screened for protein expression after induction with 500 μM IPTG and incubated for 5 h at 37° C. For protein purification, 1 L of culture was induced with 500 μM IPTG at 37° C. for 5 h. The cells were pelleted by centrifugation at 4° C. and frozen at −80° C. The cells were thawed and resuspended in the lysis buffer (50 mM Tris pH 9.0, 500 mM NaCl). The buffer pH was kept high to avoid self-splicing of the intein during the purification process. The cells were lysed by sonication and centrifuged at 20,000 rpm for 30 min at 4° C. The lysates were loaded onto a nickel-nitrilotriacetic acid (Ni-NTA) column. The column was washed with lysis buffer containing 5 mM imidazole. The protein was eluted with 250 mM imidazole, dialyzed against the buffer containing 50 mM Tris pH 9.0, 150 mM NaCl and stored at-80 for further use.

The hedgehog FRET protein C-Hh-Y was expressed and purified similarly, except that the protein was expressed in E. coli strain LMG194, induced with arabinose at 16° C. overnight, and the purified protein was stored in a storage buffer containing 10% glycerol and 4 mM TCEP.

The Cne and Cga-Prp8-inteins were expressed and purified by Ni-NTA column followed by gel filtration as described previously.

For the split nanoluciferase-Prp8 intein, in order to block the free cysteine residues, 5,5′-dithiobis-(2-nitrobenzoic) acid (DTNB) was added to 1:10 molar ratio and incubated at 4° C. for 5 min and dialyzed in 20 mM Tris, 150 mM NaCl, pH 9.0 and stored at −80° C. till further use.

For Cys-free split GFP-Prp8 intein, as the expressed protein was insoluble, the supernatant was discarded; and the pellet was resuspended in wash buffer 1 (50 mM Tris/HCL, 0.5% Triton X 100, 1 mM EDTA, 100 mM NaCl, 1 mM DDT added fresh, pH 9.0) and homogenized using a homogenizer (Wheaton, NJ USA). The suspension was centrifuged at 20,000 rpm for 20 min, and the supernatant was discarded. The washing process was repeated thrice with wash buffer 1. The pellet was resuspended and homogenized in buffer 2 (2 M urea, 2 M NaCl, 50 mM Tris/HCl, pH 8.0, 1 mM DTT added fresh). The suspension was centrifuged at 20,000 rpm for 20 min, and the supernatant was discarded. The pellet was dissolved in 8 M urea and centrifuged at 20,000 rpm for 20 min. The protein was renatured by dialyzing against buffer 3 (20 mM Tris HCl, 150 mM NaCl, pH 9.0), and purified on Ni-NTA column. After the column was washed with buffer 3 without and with 5 mM imidazole, the protein was eluted with 200 mM imidazole in buffer 3. DTNB was added to the eluted protein at a 1:10 molar ratio to block free cysteine, incubated at 4° C. for 5 min, and dialyzed against buffer 3. The protein was aliquoted and stored at −80° C. till use.

HTS assay: A resazurin-based growth assay was developed for primary screening, employing two strains of Cne, the Cne-WT with natural intein and Cne-Mut, the mutant strain without intein. The assay was first standardized in 96 well formats and later optimized for 384 well plates. The HTS was done at ICCB-Longwood screening facility, Harvard University, Boston, USA. For HTS, the fungal cells were grown in SD broth overnight and diluted in SD broth to an optical density (OD) 0.5 at 600 nm. The cell suspension is further diluted 1:10 in RPMI 1640 media with 0.165 M MOPS, 2% glucose (RPMI-MOPS) so that the final OD will be 0.05. From this cell suspension, 20 μl of the was added to each well of the 384-well assay plate (black, clear bottom) containing test compounds. Plates with test compounds were prepared by adding 20 μl of RPMI-MOPS media to each well using a liquid dispenser (Thermo Multidrop Combi), followed by transferring 1 μl of the test compound to all the 22 columns in the assay plate using robots. DMSO as vehicle control or 0.8 UM AmB as positive control were added manually to the remaining columns using a multichannel pipette. After the addition of fungal cells, the plates were incubated in a humidified incubator at 30° C. for 24 h. The 10× stock solution of resazurin in PBS was diluted in PBS and RPMI-MOPS to 1×. Resazurin (10 μl) was added to each well using a liquid dispenser (Multidrop™ Combi, Thermofisher Scientific) to a final concentration of 44 UM and further incubated at 30° C. overnight. The conversion of resazurin to resorufin by live cells were estimated by reading fluorescence at Ex 550 nm and Em 590 nm using a plate reader (EnVision, Perkin Elmer). Additionally, the absorbance was also read at 600 nm using the plate reader (EnVision, Perkin Elmer). The compound library includes known bioactive, commercial libraries, and academic molecules. The ratio of fluorescence reading in Cne-WT and Cne-Mut was used to advance positive compounds.

Molecules that showed an inhibition ratio of CneMut/CneWT of 1.5 or above and had minimum inhibition of 50% in Cne-WT were selected for dose-response assay to determine the IC₅₀. Concentration series (0.02 to 50 UM) of test compounds and equalizing volume of DMSO were dispensed using a liquid handler (D300 e, Hewlett Packard) to the 384 well plates. Following the addition of Cne cell suspension, the assay plates were incubated for 24 h, and fluorescence readings of resorufin were taken as explained for single concentration screening.

Minimum inhibitory concentration: The MIC of CMN was determined by the microdilution method in 96 well (clear, round bottom) plates. Serial double dilution of the CMN was done in the RPMI-MOPS medium. Freshly grown fungal culture from the SD agar plate was resuspended in sterile water and diluted to get 0.1 OD at 600 nm. This suspension was diluted (1:500) in RPMI-MOPS, and 100 μl was added to wells containing 100 μl of CMN. The plates were incubated at 30° C. for 48 h. Plates were checked for growth by visual observation. For Afu, resazurin was added to 44 UM and incubated for 12 h at 37° C. before noting a change in color from blue to pink by live cells. MIC is defined as the minimum concentration at which there is no growth when observed visually.

Phagocytosis assay: Mouse monocyte/macrophage-like cells RAW 264.7 were seeded in the six-well plate in DMEM media with 10% FBS and incubated at 37° C. in a CO₂ incubator. Once it reached 70% confluently, the cells were differentiated into macrophages by replacing the culture supernatant with fresh media containing 100 nM phorbol 12-myristate 13-acetate (PMA) and incubated at 37° C. for 48 h in a CO₂ incubator. Differentiated cells in six-well plates were washed once with PBS to remove loosely bound cells. Cne-WT cells were grown overnight in the SD broth media and washed thrice in 0.1 mM PBS, opsonized with 20% human complement serum (Sigma, USA) and 20 μg glucuronoxylomannan specific antibody (clone 18B7, EMD Millipore, USA) by incubating at 37° C. for 30 min with rotating end to end. The Cne cells were washed thrice with PBS to remove unbound antibodies and serum components. The opsonized Cne cells were added to the macrophages at 1:10 ratio and incubated at 37° C. for 10 h. Cells were washed thrice with PBS to remove extracellular fungal cells, followed by the addition of fresh DMEM with 10% FBS media containing various concentrations of CMN or DMSO, and incubated in a CO₂ incubator at 37° C. for 24 h. The fungal load in the media and the cells was estimated. For tittering, 100 μl of the culture supernatant was plated onto the SD-agar plates with tenfold dilutions. The cells were washed with PBS, scraped, and harvested in PBS, homogenized with a disposable homogenizer. A volume of 30 μl was mixed with 60 μl SD media and plated onto the SD agar plates. The plates were incubated at 30° C. for 72 h, and colonies were counted.

Fungistatic/fungicidal assay: To find whether CMN is fungistatic or fungicidal, a serial 2-fold dilution of CMN was made in (RPMI-MOPS) in a 96-well plate so that each well has 100 μl. Freshly grown Cne-WT colony on SD agar plate was resuspended in sterile water to give an OD600 of 0.1. This fungal suspension was further diluted at 1:500 in RPMI-MOPS media. A volume of 100 μl of the final cell suspension was added to each well in the assay plates. Additionally, 100 μl of final cell suspension was plated onto the SD agar plate to examine the actual cell count; and colonies were counted after 3 days. The assay plates were incubated at 30° C. for 48 h in a humidified incubator. The culture wells were mixed with micropipette tips, and 20 μl of the culture along with 70 μl RPMI-MOPS was plated onto the SD agar plates (100 mm) and incubated at 30° C. for 72 h before counting the colonies. The concentration of CMN at which there was no visual growth observed was considered MIC, whereas the concentration at which there was no growth on the agar plate was considered MFC. The ratio of MFC/MIC≤4 was considered fungicidal and >4 as fungistatic.

Checkerboard assay: To find the effect of CMN in combination with known antifungals, a checkerboard assay was performed employing the microdilution method in a 96-well plate according to the clinical Laboratory Standard Institute (CLS)-M60 as published previously. A combination of CMN (0.16-10 UM) and AmB (0.005 to 5.0 μM) or 5-FC (0.031 to 32 μM or voriconazole (0.001 to 0.6 UM), or itraconazole (0.002 to 2.5 μM) was performed. Cell suspensions were added to each well and incubated at 30° C. for 48 h. The absorbance was taken at 630 nm after mixing each well with multichannel pipettes. MIC was defined as the concentration to result in a reduction of ≥90% in the absorbance as compared to the control. FIC was determined as published previously. FIC≤0.5 was considered a synergistic effect, >0.5 to 1.0 additive, >1 to 4 indifferent, and >4 antagonistic.

Split nanoluciferase assay: The dose-response of CMN in inhibition of splicing was studied using Split nanoluc-Prp8 protein. The protein was diluted into 2 nM in assay buffer 20 mM tris, 150 mM NaCl with 0.1% BSA, 2 mM EDTA), and 80 μl was added to each well of 96 well plates (white, opaque). CMN was added to the first well, and serial double dilutions were done. The plates were incubated at RT for 30 min, and 20 μl of TCEP was added to a final concentration of 100 UM. The plates were sealed to prevent evaporation. After incubating for 24 h at RT, 25 μl of the diluted (1:10,000 in assay buffer) diluted nanoluc substrate furimazine (Promega) was added. The emitted luminescence was quantified using the EnVision luminescence reader (Synergyll, Biotech). The IC₅₀ was calculated using the GraphPad prism version 9.

Split GFP assay-SDS-PAGE: The cfSplit GFP-PRP8 protein at 30 M concentration was incubated with varying concentrations of CMN or DMSO for 30 min at RT in a reaction volume of 50 μl. TCEP to a final concentration of 1 mM was added to induce splicing and further incubated at RT for 24 h. An aliquot was mixed with an equal volume of 2×SDS-PAGE denaturing sample buffer. The mixture was loaded onto 12% SDS-PAGE gel without boiling. The gel, without Coomassie blue staining, was scanned with UV using Chemidoc (Bio Rad). The band densities were quantified using image lab software.

Western blot-in vivo splicing in Cne: To see the effect of CMN on the Prp8 intein splicing in vivo, freshly grown Cne-WT were diluted in the SD broth to 0.2 OD at 600 nm. SD broth (6 ml) was taken in 50 ml tubes, followed by the addition of CMN to a concentration of 2.4 μM. A serial double dilution was made by transferring 3 ml of the SD broth with CMN to the next tube containing 3 ml of the SD broth. DMSO was used as a control. An equal volume of freshly grown Cne-WT culture (0.2 OD at 600 nm) was added to obtain the final absorbance of 0.1 OD. The culture tubes were incubated in a shaker for 18 h at 30° C. Cells were harvested by centrifugation at 5,000 rpm, resuspended in 0.1 mM PBS and lysed using the bead lyser Bead Mill 24 (Fisherbrand). The lysed cells were centrifuged at 4° C., 10,000 rpm to separate cell debris. The lysate was mixed with an equal volume of 2×SDS-PAGE loading buffer, boiled, and loaded onto 12% SDS-PAGE. The separated proteins were transferred to a 0.2 μm nitrocellulose membrane. The membrane was blocked with 5% milk protein in PBST and incubated overnight with 1:5,000 dilution (in blocking buffer) of rabbit serum against recombinant Cga-Prp8-intein. After washing 3 times with PBST, 1:5,000 diluted secondary antibody goat anti-rabbit IgG-HRP (Sigma, A-6154) was added, incubated for 45 min, and washed as explained earlier. Signal was developed using chemiluminescent substrate (Super Signal West Pico plus, Thermo scientific); and images were captured using the ChemiDoc imaging system (Bio-Rad). Intensities of bands were quantified by image lab software and compared with that of control.

Thermal shift assay: Using recombinant Cne-Prp8 intein, the binding of CMN to intein protein was demonstrated by thermal shift assay. The reaction mixture (50 μl) consists of 3 M protein, 2× cyprus orange, 1.2 UM of CMN, or an equal volume of DMSO. Each treatment was done in quadruplicate. The mixture was incubated at room temperature for an hour before transferring to the QuantStudio 5 real-time PCR system (Applied Biosystems) and run with an increasing temperature from 20° C. to 90° C. The temperature at which there was 50% of the peak fluorescence (50% denaturation) was considered as Tₘ. The analysis was done using the Thermal Shift Assay software (Thermo Fischer Scientific). A graph was drawn using the GraphPad prism Version 9.1.0 after normalizing the fluorescent values. The treatment groups were compared with Student's t-test.

Microscale thermophoresis: The binding of CMN to intein was further demonstrated by the MST assay. Recombinant Cga-intein protein was diluted in assay buffer (20 mM HEPES, 150 mM NaCl, 0.5% Tween) to 400 nM. To label the protein, 100 μl 400 nM protein was mixed with an equal volume of 100 nM fluorescent dye (Monolith His-Tag Labeling Kit RED-tris-NTA 2nd Generation (Nano Temper) in assay buffer and incubated at room temperature for 30 min. After centrifugation at 15,000 rpm for 10 min at 4° C., the supernatant was transferred to a new tube. Parallelly, a stock of CMN in DMSO was diluted in the assay buffer to a concentration of 600 UM followed by serial 2-fold dilutions assay buffer in 16 tubes such that each tube has 10 μl CMN. From the above-labeled protein, 10 μl was added to each tube containing 10 μl of CMN and further incubated for 30 min. The solution was given a quick spin, loaded onto capillary tubes, and then to the MST instrument (Monolith NT.115). The assay was repeated three times, average K_{D} values were calculated, and graphs were obtained using the MO affinity analysis software.

Hh cholesterolysis assay: Cholesterolysis assay was done as per published methodology with modifications. Briefly, the recombinant C-Hh-Y protein was diluted in assay buffer (50 mM Bis-Tris, 5 mM EDTA, 100 mM NaCl, 0.4% Triton X-100) to a final concentration of 30 μM. With 50 μl reaction volume, a fixed volume of DMSO or CNM with varying concentrations (final 6.25 to 200 UM) were added and incubated at RT for 30 min. Cholesterolysis was induced by adding 1 μl stock solution of cholesterol in ethanol to each tube to attain a final concentration of 200 UM. After incubating at RT for 22 h, 20 μl of the reaction mixture was mixed with an equal volume of reducing SDS-PAGE loading buffer and loaded onto the 12% SDS-PAGE gel, followed by coomassie blue staining. Gel images were taken using the ChemiDoc imaging system (Bio-Rad). Band densities were analyzed using the Image lab software and compared with Student's t-test using the GraphPad Prism software version 9.1.0.

Cytotoxicity studies: Cytotoxicity of CMN was determined to select the concentration range of the compound in the assays. Approximately 2×104 A549 cells in MEM media with 10% FBS were seeded per well of 96 well plates and incubated at 37° C. to reach 70% confluent. Media was replaced with fresh media containing various concentrations of the CMN or DMSO. The plates were incubated for 48 h before the addition of 10 μl water-soluble MTT reagent (Cell counting kit-8, Glpbio Technology) to each well and incubated at 37° C. for 3 h. Absorbance was read at 450 nm using the plate reader (Synergy H1, Biotek); the concentration at which there was a 50% reduction in absorbance (CC₅₀) was calculated by GraphPad prism 9.1.0.

The CC₅₀ of CMN in RAW 264.7 cells were also determined. Cells were seeded in 96 well plates at 1.5×10⁴ cells/well of 96 well plates in DMEM media and incubated at a CO₂ incubator at 37° C. for 24 h. For differentiation to macrophages, the media was replaced with fresh media containing 100 nM PMA and further incubated for 48 h in a CO₂ incubator. For cytotoxicity assay, the media was replaced by fresh media containing various concentrations of CMN or DMSO and incubated for 48 h, and carried out MTT assay as explained above for A549 cells.

Toxicity studies in mice: A preliminary study of toxicity to determine the optimum dose level of CMN was conducted in adult BALB/c mice. Mice were randomly grouped (n=2) into 5 groups, vehicle control (VC), CMN 5 mg/kg, CMN 10 mg/kg, CMN 20 mg/kg, and CMN 40 mg/kg. The formulation of the compound was done by dissolving the CMN in absolute DMSO and diluted in corn oil (Sigma C-8267) to a final 10% DMSO and 90% corn oil. CMN was dosed to mice by oral gavage 70 μl/mouse daily for 7 days. The mice were observed twice daily for general health, movement, activity, and coat condition and scored. Food and water were ad libitum. On day 7, the mice were euthanized, and necropsy was done to see any changes in the organs as compared to the normal mice by visual observation.

Pharmacokinetics in mice: C57BL/6 mice (male, 2-3 months old) were obtained from breeding stocks maintained at the University of Arizona. All mice were housed under conditions of controlled temperature (22° C.) with the on-off light cycle, food, and water provided ad libitum and were fasted for 12 h before administration of the compound. Mice (n=5) were dosed with CMN 40 mg/kg (in 90% corn oil and 10% DMSO). Blood samples were collected from the tail vein using heparinized capillary tubes at 0, 0.5, 1, 2, 4, 8, 12, 16, 24, and 36 h after oral gavage. Plasma was separated and stored at −30° C. until use. For extraction of the compound, 5 μl of plasma from each mouse were mixed with 30 μL methanol and 10 μl of internal standard (IS; UAWJ102 50 ng/ml). The mixture was vortexed for 30 seconds and diluted with 760 UL Milli Q water, and loaded onto an ISOLUTER C18 SPE Columns (1 ml/100 mg, Biotage, Salem, NH) pre-conditioned with 1 ml methanol followed by 1 ml water, and the cartridge was then washed with 1 ml water. The analytes were finally eluted from the cartridges with 1 ml methanol containing 5% ammonia solution, dried with nitrogen, and reconstituted in 100 μl methanol.

The analyte was detected and quantified using LC-MS (Agilent 1290 UPLC system Agilent Technologies, Santa Clara, CA) and a Sciex Qtrap6500+ Mass Spectrometer (AB SCIEX, Framingham, MA). Analytes were separated on an EclipsePlus C18 column (2.1×50 mm, 1.8 μm, Agilent) at a temperature of 35° C., with mobile phase A containing 0.1% formic acid (v/v) in water and mobile phase B containing 0.1% formic acid (v/v) in methanol. Elution was at a flow rate of 0.2 mL/min as follows: 10% B (0-0.5 min), 10% B→95% B (0.5-3.5 min), 95% B (3.5-7 min), 95% B+10% B (7-7.1 min), 10% B (7.1-10 min). The MS was operated in the positive ion mode, using electrospray ionization. The ion spray voltage and temperature were set at 5000 V and 500° C., respectively. Curtain gas, ion source gas 1, and ion source gas 2 were set at 25, 50, 50 psi, respectively. CMN and the internal standard (IS, UAWJ102) were detected using Multiple Reaction Monitoring (MRM), with a dwell time of 200 msec per transition, at m/z 524.4/488.2 and 289.1/139.1, respectively. Retention times for CMN and the IS were 5.9 and 3.1 min, respectively. For quantitative analysis of CMN, standards (5 to 1000 ng/ml in 10 μl methanol), along with 10 μl IS (at 50 ng/ml in methanol), were added to 5 μl of blank plasma of the mouse for obtaining a calibration curve.

Efficacy studies in the mouse: The in vivo studies were done in a mouse model with the approval of IACUC, Wadsworth Center, Albany, NY. Male and female BALB/c mice 4-5-week-old were purchased from Jackson laboratory and quarantined for a week before shifting to the experimental room. The mice were grouped randomly with a male: female ratio 1:1, ears were punched as identification code. The formulation of the compound was done by dissolving the CMN in absolute DMSO and diluted in corn oil. The formulations were done daily, just before the dosing. On day 0, mice were given CMN 20 mg/kg in 70 μl or the vehicle control intraperitoneally. Following this, the mice were anesthetized and infected with Cne-H99 cells (107 cells in 30 μl PBS/mouse) intranasally. From day 1 to day 5, mice were treated with only CMN. Mice were weighed every day, and visual observations were made for activity, movement, coat condition, etc. Food and water were ad libitum. On day 5, the mice were anesthetized and euthanized. Lung tissues were dissected out, weighed, and homogenized in 1 ml RPMI-MOPS media using a glass homogenizer. After log dilution of the tissue suspension in RPMI-MOPS, 100 μl from each dilution was plated onto SD agar plates with antibiotics (20 μg/ml penicillin, 40 μg/ml streptomycin, 40 g/ml gentamicin, 25 μg/ml chloramphenicol). The plates were incubated at 30° C. for 3 days before counting the colonies. The counts were normalized with the weight of the lung tissue taken during the necropsy. The fungal load in the groups was compared with Student's t-test, and p<0.05 was considered as significant.

Statistical analysis: Unpaired, Student's t-test was used to compare the significance between treatment groups. p<0.05 was considered as significant. GraphPad Prism version 9.1.0 was used for drawing graphs and calculating IC₅₀.

CMN specifically inhibits the Prp8-intein containing fungi Cne and Cga: To identify intein-specific inhibitors, an HTS cell viability assay was standardized using resazurin in 384-well format. The fluorescence-based assay gave a signal-to-noise ratio of 5.62. The Z′ factor and coefficient of variation (CV) for the assay were 0.7 and 8.0, respectively. The screening was done both in Cne-WT, and the inteinless Cne-Mut developed previously, which will help to select molecules specific for the intein-containing strain but not for the inteinless strain. Primary screening of test compounds was done at a final concentration of 50 UM in a resazurin-based HTS growth assay at the ICCB-Longwood screening facility, Harvard University, Boston, USA. The compound libraries include known bioactive, academic compounds, and commercial libraries. A total of 98,440 compounds were screened in the primary screening. Compounds were advanced if they showed more than 50% inhibition in Cne-WT, and the ratio of Cne-Mut to Cne-WT was ≥1.5-fold. Out of the total molecules screened, 391 molecules were advanced for IC₅₀ estimation in both strains of Cne. Among the selected compounds, calcimycin (CMN) (FIG. 1A) showed a 2.5-fold difference in IC₅, in Cne-WT and hence was selected for further characterization.

The antifungal activity of CMN was tested against various fungal species. The MIC values of CMN were 1.5 μg/ml and 25.1 μg/ml in Cne-WT and Cne-Mut, respectively (Table 1). CMN also inhibited the growth of Cga with MIC of 1.5 μg/ml, whereas no inhibition was observed for CMN against a natural inteinless cryptococcal sp. Cam that is closely related to Cne and Cga. With Afu, there was no inhibition up to 26 μg/ml of CMN. These data suggest that CMN is specific to the Prp8-intein containing fungi Cne and Cga, and to a lesser extent to Afu, but not to the intein-free fungus such as Cam.

TABLE 1
Antifungal activity of CMN. MIC were determined using microdilution
method. The visual observation was done after 48 h of incubation
at 30° C. MIC is defined as the minimum concentration
of the drug where there was no growth observed visually
(Additional details are with FIG. 6).

| Species (Strain) | MIC μg/ml (μM) | |
|---|---|---|
| C. neoformans (Cne-WT) | 1.5 | (3.0) |
| C. neoformans (Cne-Mut) | 25.1 | (48) |
| C. gattii (NIH444) | 1.5 | (3.0) |
| C. amylolentus | >26.1 | (>50) |
| A. fumigatus | >26.1 | (>50) |

CMN inhibits the growth of intracellular Cne in macrophages: Cne is an intracellular pathogen, with alveolar macrophages and innate phagocytes as the first line of defending cells in the lung. Reducing intracellular infection of Cne in macrophages is essential to treat cryptococcal infections in humans. Therefore, the effect of CMN in inhibiting intracellular infection of Cne in macrophages differentiated from macrophage-like RAW 264.7 cells was evaluated using a macrophage intracellular infection model (FIGS. 1B, 1C, and 1D). The results showed that CMN significantly reduced the growth of intracellular Cne in macrophages at 0.6 μg/ml (FIG. 1C) and released Cne at 0.3 μg/ml (FIG. 1D). The reduction was dose-dependent in both intracellular (23%, 50%, 72% at 0.3, 0.6 and 1.3 g/ml) and released Cne (41%, 59%, 69% inhibition at 0.3, 0.6 and 1.3 μg/ml respectively). Overall, the results indicate that CMN inhibits the Prp8-intein containing cryptococcal fungi Cne intracellularly and extracellularly.

CMN is fungistatic and synergizes with AmB: To investigate the nature of inhibition, the minimum fungicidal concentration (MFC) of CMN was determined in Cne as explained in material and methods (FIG. 6). After culturing the Cne-WT with various concentrations of CMN for 48 h, 50 μl of the culture supernatants were plated onto the SB agar plates. Colonies were counted on day 3 to determine the MFC. There was growth up to 26 μg/ml CMN, whereas the MIC was 1.5 μg/ml. The ratio of MFC/MIC was >4. Therefore, CMN is considered fungistatic. (FIG. 6).

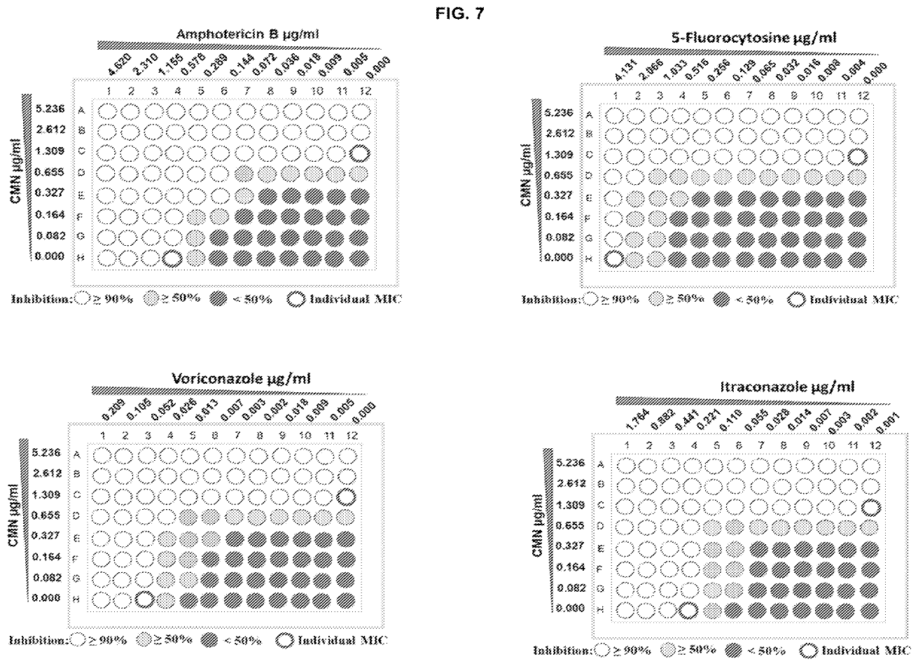

As the fungistatic drug is best used together with fungicidal compounds to treat fungal infection, a checkerboard assay was performed to determine the fractional inhibitory concentration (FIC) to evaluate synergy between calcimycin and existing antifungals. CMN showed a synergistic effect with AmB (FIC index of 0.49), additive effect with 5-FC (FIC index 0.99), indifferent with voriconazole and itraconazole (FIC index 1.50 and 1.25, respectively) (Table 2 and FIG. 7).

TABLE 2
Combinational effect of CMN with known antifungals.
MIC were determined by checkerboard assay. FIC index value
of ≤0.5 synergy, ≥0.5 to 1 is additive, >1 to 4 is indifferent
(Additional details are with FIG. 7).

| Well | | MIC μg/ml | | FIC | |
|---|---|---|---|---|---|
| position | Compounds | Comb | Alone | index | Effect |
| E6 | CMN | 0.33 | 1.31 | 0.49 | Synergy |
| | Amphotericin B | 0.14 | 0.6 | | |
| D2 | CMN | 0.65 | 1.31 | 0.99 | Additive |
| | 5-Fluorocytosine | 2.06 | 4.13 | | |
| D4 | CMN | 0.65 | 1.31 | 1.50 | Indifferent |
| | Voriconazole | 0.05 | 0.05 | | |
| E4 | CMN | 0.33 | 1.31 | 1.25 | Indifferent |
| | Itraconazole | 0.22 | 0.22 | | |

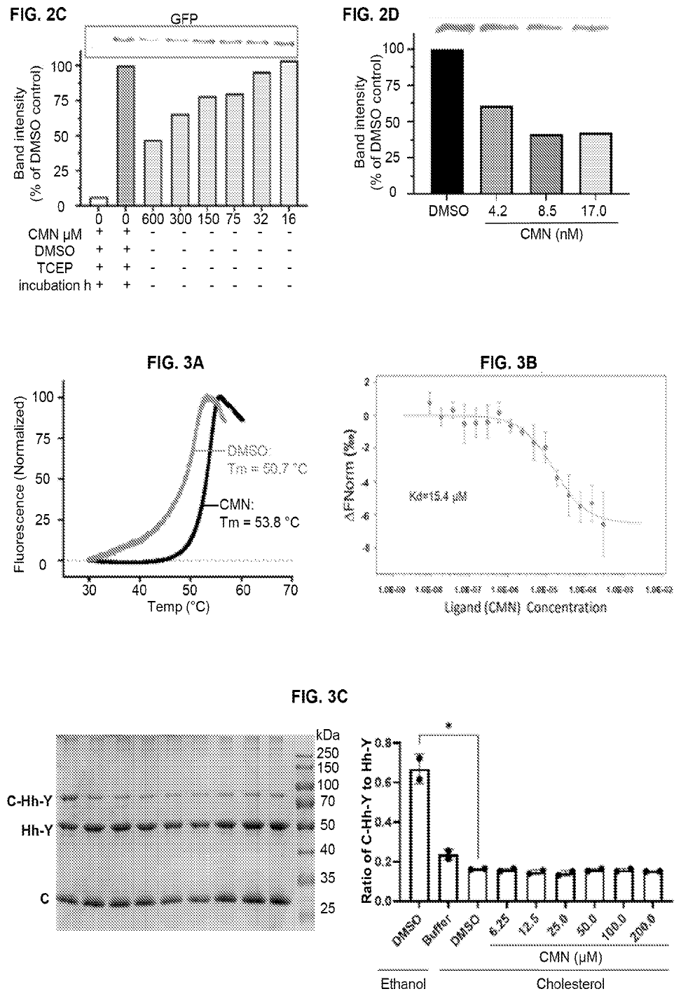

Inhibition of Prp8 intein splicing in vitro and in vivo: One drawback of firefly luciferase is that it may be sensitive to redox reactions due to four cysteine residues. In order to overcome this issue, a Cys-free split nanoluciferase (cfNanoLuc) was developed based on a Cys-free NanoBit nanoluciferase. The His-tagged CnePrp8 intein was inserted between NanoBit residues 156 and 157 or between residues 52 and 53. Because of the higher solubility, the fusion protein that has intein inserted at 156-157 was used for assays. Upon addition of tris(2-carboxyethyl) phosphine (TCEP) and NanoBit substrate, significant luminescence was detected (FIG. 2A). This indicates the splicing of Prp8 intein from the N-NanoBit-Prp8 intein-NanoBit-C, leading to the reconstitution of non-interrupted NanoBit. Theoretically, inhibition of the Prp8 intein splicing by inhibitors will keep NanoBit in two non-interacting fragments interrupted by the Prp8 intein, resulting in inactive NanoBit and decreased luminescence. Using cisplatin, a known Prp8 intein inhibitor, we showed that cisplatin efficiently abolished TCEP-trigged luminescence increase, indicating that the Prp8-NanoBit splicing is successfully inhibited by cisplatin (FIG. 2A) with an IC₅₀ of 1.41 μM. The results validated that the Prp8-NanoBit splicing assay could be used to characterize inhibitors targeting the Prp8 intein splicing.

The Prp8-Nanobit splicing assay was used to investigate whether CMN inhibited the Prp8 intein splicing in vitro. As shown in FIG. 2B, CMN treatment inhibited TCEP-trigged luminescence increase in a dose-dependent manner. The IC₅₀ of CMN was 8.7±0.7 UM (4.6 μg/ml) in the NanoBit splicing assay (FIG. 2B). Similarly, the splicing of split GFP-Prp8 intein was inhibited with CMN dose-dependently compared to that of the DMSO control when analyzed on SDS-PAGE (FIG. 1C). In split GFP-based splicing assay, CMN reduced the level of splicing although at higher concentrations. This may be because a higher concentration of the protein (30 μM) must be used to obtain a visible band of GFP in SDS-PAGE.

Next, whether CMN inhibited the Prp8 intein splicing in vivo in Cne was investigated. An effective way to quantify the Prp8 intein splicing in vivo is to monitor the spliced Prp8 intein using an anti-Prp8 intein antibody or serum. Previously, a rabbit polyclonal antibody serum specifically recognizing the Prp8 inteins of Cne and Cga was developed and used in the characterization of the Prp8 intein splicing in vivo. Using this anti-Prp8 intein serum, in the in vivo Prp8 intein splicing assay in Cne-WT, there was a dose-dependent reduction in spliced intein (approximately 20 kDa) when detected by serum against the CgaPrp8 intein in western blot (WB) assay (FIG. 2D). A band at around 130 kDa was also detected. These data suggest that CMN inhibits the Prp8 intein splicing both in vitro and in vivo.

Target binding and specificity: To further investigate the mode of action of CMN in inhibition of the Prp8 intein splicing, a protein TSA was carried out, as described in material and methods. The protein denaturation Tₘ values were determined for the recombinant CnePrp8 intein in the presence and absence of CMN. With the DMSO control, the Tₘ value was 50.8±0.2° C., whereas with 1.2 UM CMN, Tₘ was increased to 53.8±0.2° C. (FIG. 3A). The ΔTₘ difference of 3° C. between CMN and DMSO indicates that CMN binds to the Prp8 intein. The target binding of CMN was also confirmed using MST. The average K_{D} of three repeats was 15.4 UM (FIG. 3B).

Although human proteins do not carry intein elements, Hh protein cholesterolysis is mechanistically similar to intein splicing. Therefore, it is necessary to investigate whether CMN can lead to an off-target effect. Using a cyan fluorescent protein (CFP)-Hh-Yellow fluorescent protein (YFP) (C-Hh-Y) FRET substrate, CMN at up to 200 UM did not change the density of spliced product in the drosophila hedgehog-cholesterolysis assay in vitro (FIG. 3C). The results indicate that CMN specifically inhibits the Prp8 intein splicing but not human hedgehog cholesterolysis.

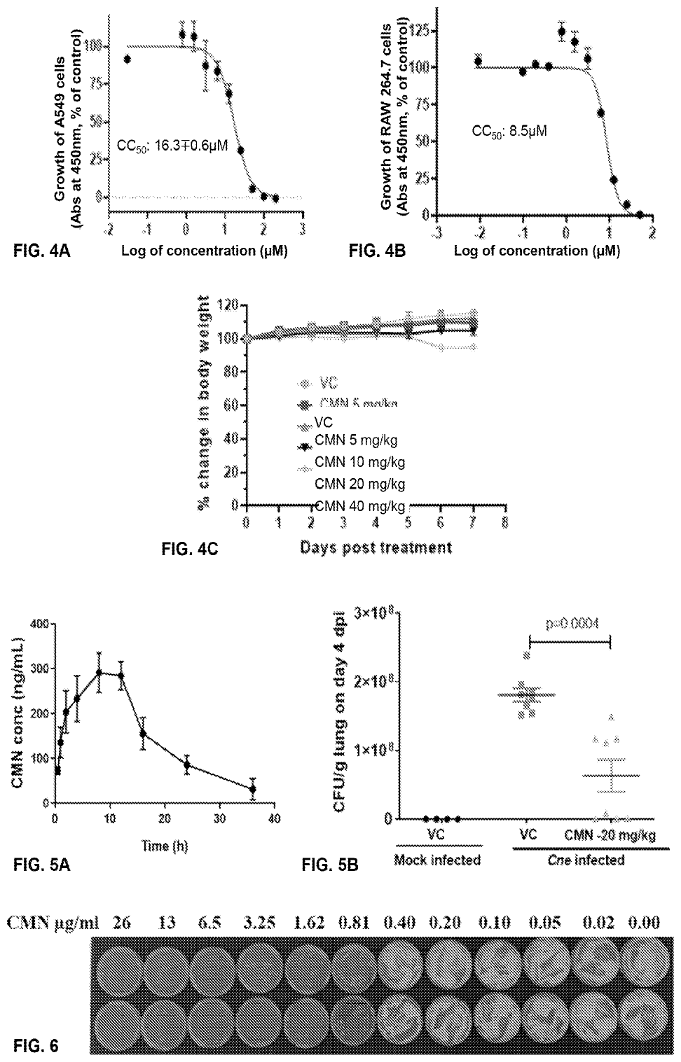

Toxicity studies in vitro and in vivo: The potential cytotoxicity of CMN on human cell lines was determined by using a WST-8 cell viability assay as described herein. The cytotoxicity CC₅₀ values of CMN were 16.3 UM (8.6 μg/ml) in human alveolar basal epithelial A549 cells (FIG. 4A) and 8.6 UM (4.5 μg/ml) in the PMA-differentiated murine macrophage RAW 264.7 cells (FIG. 4B).

To further investigate the toxicity of CMN, a preliminary 7-day in vivo toxicity study was performed using BALB/c mice. There was no significant change in activity, the appearance of fur, weight loss with daily oral gavage at 5, 10, 20 mg/ml. At 40 mg/ml, the activity of mice was slower for 1-2 h post-dosing, and there was an 8% loss of body weight by day 7 (FIG. 4C), and no other morbidity was observed (Table 3).

TABLE 3
Mouse toxicity studies: Scoring was done as explained in material
and methods. Scoring criteria: Posture (0 = normal, 1 =
slightly hunched, 2 = moderately hunched, 3 = severally
hunched). Coat condition (0 = normal/groomed, 1 = rough,
2 = ruffled/unkempt). Activity/Alertness (0 = normal, 1 =
reduced exploratory activity, 2 = slow-moving, dull or
depressed, 3 = not moving). Movement/Gait (0 = Normal,
1 = slight incoordination/decreased righting response, 2 =
tiptoe walking or altered gait involving 1 limb, 3 = staggering
or paralysis or tremors of 2 or more limbs. VC: Vehicle control.

| | CMN mg/kg BW | | | | | | | | | |
|---|---|---|---|---|---|---|---|---|---|---|
| Parameters | 0 (VC) | | 5 | | 10 | | 20 | | 40 | |
| Posture | 0 | 0 | 0 | 0 | 0 | 0 | 0 | 0 | 0 | 0 |
| Coat condition | 0 | 0 | 0 | 0 | 0 | 0 | 0 | 0 | 0 | 0 |
| Movement | 0 | 0 | 0 | 0 | 0 | 0 | 0 | 0 | 1 | 1 |
| Activity/alertness | 0 | 0 | 0 | 0 | 0 | 0 | 0 | 0 | 0 | 0 |
| Movement | 0 | 0 | 0 | 0 | 0 | 0 | 0 | 0 | 0 | 0 |
| Seizure | 0 | 0 | 0 | 0 | 0 | 0 | 0 | 0 | 0 | 0 |
| Death | 0 | 0 | 0 | 0 | 0 | 0 | 0 | 0 | 0 | 0 |

Pharmacokinetics: Although CMN is a candidate drug currently in clinical investigation, there were no reports of pharmacokinetic (PK) studies of CMN in the mouse. Therefore, PK studies were carried out by dosing mice with 40 mg/kg of CMN by oral gavage. CMN was absorbed slowly in mice and reached its plasma peak concentration Cₘₐₓ of 301 ng/ml at Tmax of around 10 h. The clearance of CMN was also relatively slow, with a t_{1/2} of 7.6 h (Table 4, FIG. 5A).

TABLE 4
Pharmacokinetic parameters of CMN in mice. CMN was administered
to adult male C57BL/6 mice at 40 mg/kg BW by oral gavage as explained
in the methods. Values represent means ± S.D. (n = 5).

| Parameter | Unit | Value |
|---|---|---|
| Cmax | ng/ml | 301 ∓ 38 |
| Tmax | h | 9.60 ∓ 2.19 |
| t½ | h | 7.62 ∓ 2.50 |
| AUC 0-inf_obs | ng/ml*h | 5,822 ∓ 1,157 |
| Vz/F_obs | 1/kg | 74.6 ∓ 12.2 |
| C1/F_obs | 1/kg/h | 7.08 ∓ 1.31 |

CMN reduces fungal burden in a Cne mouse model: After determining the in-vitro activity and PK properties, the in vivo antifungal efficacy was determined using a cryptococcal pulmonary infection model. In the efficacy studies, mice were infected intranasally with either PBS (mock) or 10⁷ Cne cells/mouse. All mice were treated orally with vehicle or CMN at 20 mg/Kg body weight (BW) once daily for 5 days. On day 5, mice were euthanized to determine the fungal burden in the lungs. The Cne load in the vehicle control group was 1.8×10⁸ (SEM±9.8×10⁶) CFU/g lung tissue, whereas the CMN-treated group showed a 3-fold reduction in fungal burden with 6.3×10⁷ (SEM+2.3×10⁷) CFU/g lung tissue (FIG. 5B). Neither fungal colonies nor abnormalities in mice were observed in the mock-infected group. Overall, these results indicate that CMN effectively reduces fungal burden in vivo in a mouse model.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only, and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of treating a disease caused by a fungus in a subject in need thereof, wherein the fungus comprises inteins, the method comprising administering a therapeutically effective amount of calcimycin to the subject.

2. (canceled)

3. The method of claim 1, wherein the fungus is from the genus Cryptococcus; wherein the fungus is Cryptococcus neoformans (Cne) or Cryptococcus gattii (Cga).

4.-5. (canceled)

6. The method of claim 3, wherein the disease is cryptococcosis.

7. The method of claim 1, wherein calcimycin inhibits pre-mRNA processing factor 8 (Prp8) proteins.

8. The method of claim 1, wherein the fungus is from the genus Candida; wherein the fungus is Candida fermentati, Candida parapsilosis, Candida krusei, or Candida albicans.

9. (canceled)

10. The method of claim 8, wherein the disease is candidiasis.

11. (canceled)

12. A method of treating a disease caused by a microbe in a subject in need thereof, wherein the microbe comprises inteins, the method comprising administering a therapeutically effective amount of calcimycin to the subject.

13. The method of claim 12, wherein the microbe is a fungus.

14. The method of claim 13, wherein the fungus is from the genus Cryptococcus; wherein the fungus is Cryptococcus neoformans (Cne) or Cryptococcus gattii (Cga).

15.-16. (canceled)

17. The method of claim 14, wherein the disease is cryptococcosis.

18. The method of claim 13, wherein the fungus is from the genus Candida; wherein the fungus is Candida fermentati, Candida parapsilosis, Candida krusei, or Candida albicans.

19. (canceled)

20. The method of claim 18, wherein the disease is candidiasis.

21. The method of claim 12, wherein the microbe is a bacterium.

22. The method of claim 21, wherein the bacterium is from the genus Mycobacterium; wherein the bacterium is Mycobacterium tuberculosis (Mtb).

23.-30. (canceled)

31. A method of inhibiting intein splicing in a microbe comprising inteins, the method comprising introducing to said microbe a composition comprising calcimycin.

32. The method of claim 31, wherein the microbe is a fungus.

33. The method of claim 32, wherein the fungus is from the genus Cryptococcus; wherein the fungus is Cryptococcus neoformans (Cne) or Cryptococcus gattii (Cga).

34.-35. (canceled)

36. The method of claim 31, wherein calcimycin inhibits pre-mRNA processing factor 8 (Prp8) proteins.

37. The method of claim 32, wherein the fungus is from the genus Candida; wherein the fungus is Candida fermentati, Candida parapsilosis, Candida krusei, or Candida albicans.

38.-39. (canceled)

40. The method of claim 31, wherein the microbe is a bacterium; wherein the bacterium is from the genus Mycobacterium; wherein the bacterium is Mycobacterium tuberculosis (Mtb).

41.-68. (canceled)


SEQUENCE LISTING (1 sequence)

SEQ ID NO: 1 - protein, 18 aa, synthetic construct
GHHHHHHEQKLISEEDLG